(19) United States
(12) Patent Application Publication
(10) Pub. No.: US 2003/0066523 A1
(43) Pub. Date: Apr. 10, 2003

(54) Sugar additive blend useful as a binder or impregnant for carbon products

(75) Inventors: Irwin Charles Lewis, Strongsville, OH (US); Terrence Anthony Pirro, Cleveland, OH (US)

Correspondence Address: James R. Cartiglia, UCAR Carbon Company Inc., Brandywine West, 1521 Concord Pike, Suite 301, Wilmington, DE, 19803, US

(73) Assignee: UCAR Carbon Company Inc., Nashville, TN (US)

(21) Appl. No.: 10/185,058

(22) Filed: Jun. 28, 2002

Related U.S. Application Data
(62) Division of application No. 09/967,734, filed on Sep. 28, 2001.

(51) Int. Cl. C07H 1/00; C13F 3/00

(52) U.S. Cl. 127/29; 536/121; 536/122; 536/1.11

(57) ABSTRACT

A sugar/additive blend useful as a binder or impregnant for carbon products. Simple sugars as well as sucrose are combined either in solution or in solid form with reactive additives such as ammonium hydrogen phosphate, ammonium chloride and para-toluene sulfuric acid. The sugar/additive blends form more and denser carbon residue than sugar alone when subjected to pyrolysis.

### TECHNICAL FIELD

This invention relates to the use of sugars together with additives as binders or impregnating agents for carbon products. Such binders are mixed with cokes and heated to form molded bodies and/or may be used as impregnating agents which when carbonized density and strengthen the underlying carbon substrates to which they are applied.

### BACKGROUND OF THE INVENTION

Sugar is one of many precursor materials which has been suggested for use as an impregnant or binder which may be mixed with or applied to a carbonaceous material and then pyrolyzed to decompose and leave behind only carbon. According to such a procedure, solid sugar, or a solution thereof, may be used to mold a carbonaceous material such as a coke into a preform, which may then be heated to form a green coke. When used as an impregnating agent, the sugar, or an aqueous solution thereof, may be used to fill existing pores in an underlying carbonaceous preform and then heated such that the pores become filled with a carbon residue, with the result being a carbon product with enhanced density and mechanical strength.

For instance, U.S. Pat. No. 935,180 to Williamson, dated Sep. 28, 1909, and U.S. Pat. No. 963,291 to Horton, dated Jul. 5, 1910, teach the use of a solution containing a carbohydrate such as molasses to impregnate porous graphitic articles. U.S. Pat. No. 4,472,460 to Kampe, et al., dated Sep. 18, 1984, teaches the use of a liquid sugar solution to coat carbon black particles, which, upon pyrolysis, form a continuous coating of electrically conductive carbon char for use in gas diffusion electrodes. U.S. Pat. No. 3,026,214 to Boyland, et al., dated Mar. 20, 1962, teaches the use of solutions of purified sugar to impregnate carbon bodies in repeated high-temperature processing cycles. These art-described processes, which utilize sugar or other carbohydrates as carbon precursors, prefer that the sugar or carbohydrate be dissolved in either water or some other appropriate solvent.

FR 2,786,206 teaches the use of crystalline sugar as a binding agent. According to that patent 10 to 25% of crystalline sugar is mixed with 50 to 70% petroleum coke and 15 to 30% “abouts” and then carbonized to 1000° C. to produce a carbon anode. In a publication by R. J. Price and G. H. Reynolds, Proceedings of the 1997 Biennial Carbon Conference p. 520, fructose and glucose are used as impregnants to densify carbon-carbon composites. The Price and Reynolds impregnation was carried out at 20° C. above the melting point of the sugars, and then the impregnated articles pyrolyzed in air from 250-325° C. followed by carbonization at 950° C.

Sugars would be desirable as carbon binders and impregnants from an environmental standpoint in comparison to the conventional binders and impregnant materials such as pitches and phenolic resins since the major volatile by-product during curing and carbonization is water. However, upon pyrolysis sugars give very low carbon yields, generally about 18-20% compared to 50-60% for conventional binding and impregnating materials such as pitches and resins. The elemental carbon content of simple sugars is only about 40%, with the remainder being mainly oxygen along with some hydrogen. During carbonization all the oxygen and hydrogen along with about 50% of the carbon is evolved, leaving a relatively low carbon yield.

Another disadvantage of using sugars as binders and impregnants is that they undergo an exothermic polymerization over a very narrow temperature range with copious evolution of water. For example, sucrose when heated melts at about 190° C. and then polymerizes exothermically during curing at about 250° C. with the evolution of about 50-60 weight % of volatiles, which are largely water. This effect results in a weak, foamy carbon product from standard sugars.

### SUMMARY OF THE INVENTION

Accordingly, it is an object of this invention to employ sugars together with reactive additives as binding agents or impregnants for carbon products and by the use of such additives to increase the carbon yield of the sugars and thereby retain more of the intrinsic carbon and to achieve a carbon yield closer to the theoretical value of about 40% than has heretofore been possible.

As used herein, the term “sugar” is to be understood as meaning any of a number of useful saccharide materials. Included in the list of useful sugars are the mono-saccharides, disaccharides and polysaccharides and their degradation products, e.g., pentoses, including aldopentoses, methylpentoses, keptopentoses, like xylose and arabinose; deoxyaldoses like rhamnose; hexoses and reducing saccharides such as aldo hexoses like glucose, galactose and mannose; the ketohexoses, like fructose and sorbose; disaccharides, like lactose and maltose; non-reducing disaccharides such as sucrose and other polysaccharides such as dextrin and raffinose; and hydrolyzed starches which contain as their constituents oligosaccharides. A number of sugar syrups, including corn syrup, high fructose corn syrup, and the like, are common sources as are various granular and powdered forms. In general, sugars contemplated for use in the invention should be of commercial quality, although they need not be of food grade.

It is a further object of the invention to expand the temperature range during which sugars undergo a curing polymerization reaction, to lessen the foaming effect which otherwise might accompany the polymerization reaction and to thereby densify and increase the strength of the carbon derived from the sugar.

It is a further object of the invention to utilize the high solubility of sugars in water, whereby a concentrated solution of sugar in water together with an appropriate reactive additive gives an effective binder or impregnant which can be used at room temperature.

It is a still further object of the invention to utilize solid sugar/reactive additive blends directly as binders by mixing the sugar/additive blends with fillers and then heating the resulting mixture above the melt temperature of the sugar/additive blend to form artifacts by molding or extrusion, and/or to use such solid sugar additive blends as impregnating agents by heating the blends above their melt temperature and applying the molten blends to a heated carbon or graphite preform.

These and other objects are accomplished by combining sugars with selected additives as set forth herein.

Many preferred and alternative aspects of the invention are described below.

### BRIEF DESCRIPTION OF THE DRAWINGS

The invention will be better understood and its advantages more apparent from the following description of the accompanying drawings wherein:

FIG. 1 is a differential scanning calorimetry (DSC) plot demonstrating the heat flow involved in the heating of sucrose.

FIG. 2 is a thermogravimetric analysis (TGA) plot demonstrating the weight loss involved in the heating of sucrose.

FIG. 3 is a DSC plot demonstrating the heat flow involved in the heating of sucrose containing 3.7% ammonia dihydrogen phosphate.

FIG. 4 is a TGA plot demonstrating the weight loss involved in the heating of sucrose containing 3.7% ammonia dihydrogen phosphate.

### DETAILED DESCRIPTION OF THE INVENTION

The invention will be illustrated and explained in this description by reference to particular blends of additives with sucrose and fructose for use as binder and/or impregnating pitches. It will be recognized, however, that while this description is made for illustrative purposes, the invention has broader applicability and is useful in other processes and in connection with various other end uses.

The sugar additive blend of the invention as described herein can be used as a carbon precursor material such as directly as an impregnant for carbon articles, or as a binder for carbonaceous particles, or can be dissolved in a solvent such as water to provide a solution binder or impregnant.

Advantageously, the sugar is beneficially combined with from about 1 to 6% of a reactive additive such as a phosphate like ammonium dihydrogen phosphate and ammonium monohydrogen phosphate; ammonium chloride; zinc chloride; aluminum chlorate; or para-toluene sulfonic acid (PTSA). The reactive additive is a composition which, when combined with sugar, will remove water from the sugar earlier than would otherwise occur, and thus stabilize the carbon present in the sugar. The additive is preferably selected from compositions which when blended with sugar and pyrolyzed will yield predominantly volatiles, leaving little residue other than carbon.

Effective reactive additives are acids and acid salts including salts of phosphoric acid, which can accelerate the removal of OH groups as water, and the formation of double bonds in the sugar ring structure. The additive must be soluble in the desired solvent, especially water, and react with the sugar at a temperature below the normal decomposition temperature without the additive. The additive stabilizes the carbon structure in the sugar molecule by forming double bonds, thus increasing overall carbon yield. Preferably the additive is present in an amount of up to about 6% by weight, based on the amount of sugar, although there is no true upper limit to the amount of additive other those due to practical considerations. More preferably, the additive is present at a level of about 1% to about 4% by weight.

The sugar/additive blend can then be combined with a carbon filler such as petroleum coke or carbon fibers and formed into an artifact by molding or extruding at a temperature above the melt point of the sugar/additive blend. The formed artifact may then be heated to about 200° C.-250° C. to “cure” the sugar and then carbonized or graphitized at a conventional desired final temperature, as is known in the art.

The sugar/additive blend as described in the preceding paragraph can also be used as an impregnant for carbon articles by heating above the melt point of the blend and using standard impregnation procedures as are known in the art for fabricating carbon articles. However, it is preferable to carry out the impregnation using a solution of the sugar/additive blend, especially an aqueous solution. In this way, the impregnation can be carried out at room temperature using a concentrated solution of sugar while maintaining a low viscosity. For this purpose a solution preferably containing from about 25% to about 70% of sugar in water and preferably from about 1 to about 4% of additive/sugar can be prepared. Such solutions can be used to impregnate carbon bodies and then heated under vacuum to remove water followed by curing and carbonizing. The relatively low viscosity of the solution permits maximum pore penetration and subsequent carbonization will result in enhanced density and strength of the preform.

The sugar/additive solution in water as described in the preceding paragraph can also be used as a binder by adding to it the appropriate carbon filler (coke or fiber), filtering to remove the excess water and then forming the filler/sugar/additive blend by melting or extrusion above the sugar melt point. The formed artifact can be cured, such as by heating at 200-250° C., and carbonized to the desired final temperature.

One method the inventive sugar/additive blend can be used in forming a carbon article is by combining the blend with a carbonaceous source material such as a coke to form a raw mix; extruding the mix to form a preform; baking the preform blend to form a carbonized blend; and graphitizing the carbonized blend by heating to a temperature of at least about 2500° C. and maintaining it at that temperature for sufficient time to graphitize it and form a graphitic article.

Baking is preferably at a temperature of between about 700° C. and about 1100° C., more preferably between about 800° C. and about 1000° C., and functions to carbonize the binder, to give permanency of form, high mechanical strength, good thermal conductivity, and comparatively low electrical resistance. The green blend is baked in the relative absence of air to avoid oxidation. Baking should be carried out at a rate of about 1° C. to about 5° C. an hour to the final temperature. After baking, the blend may be impregnated one or more times with the inventive sugar/additive blend, or coal tar or petroleum pitch, or other types of pitches known in the industry, to deposit additional carbon in any open pores of the pin. Each impregnation is then followed by an additional baking step.

After baking the blend referred to at this stage as carbonized blend, is then graphitized. Graphitization is by heat treatment at a final temperature of between about 2500° C. to about 3400° C. for a time sufficient to cause the carbon atoms in the calcined coke and binder to transform from a poorly ordered state into the crystalline structure of graphite. Advantageously, graphitization is performed by maintaining the carbonized blend at a temperature of at least about 2700° C., and more advantageously at a temperature of between about 2700° C. and about 3200° C. At these high temperatures, elements other than carbon are volatized and escape as vapors.

Contrariwise, the raw mix can be formed using coal tar or petroleum pitch, rather than the inventive sugar/additive blend, with the sugar/additive blend used for impregnation only.

The following Examples are provided to further illustrate and explain a preferred form of the invention and are not to be taken as limiting in any regard. Under otherwise indicated, all parts and percentages are by weight.

### EXAMPLE 1

This example demonstrates the effect of different additives on the carbon yield obtained from the carbonization of sucrose.

To a 27% solution of sucrose in water, each of the various additives set forth in Table I were added at a level of 1 part additive to 27 parts of sucrose (3.7%). The viscosity of this solution was measured as about 10 cps showing it would be suitable as an impregnant for carbon articles at room temperature. The solutions were heated under vacuum at about 70° C. to remove the water and leave the solid sugar residue containing the dispersed additive. Carbon yield for the pyrolyzed sugar additive blends was measured using the modified Conradson Carbon procedure (MCC). This procedure is described on page 51, Volume II of “Analytical Methods for Coal and Coal Products”, C. Carr, Jr. Academic Press (1978). The results in Table I show that carbon yield, or amount of residual carbon, was increased by up to 88% by use of the additive. Inspection of the carbon residues which resulted from heating of the sugar/additive blends showed that without the additive, the sucrose derived carbon was extremely weak and foamy, whereas the sugar/additive carbons were generally harder and denser. The 37% carbon yield measured for the ammonium dihydrogen phosphate represents about 88% retention of the total carbon in sucrose.

TABLE I
Effect of Additives on MCC of Sucrose

| Additive (3.7%) | MCC % |
|---|---|
| None | 20 |
| NH₄H₂PO₄ | 37 |
| (NH₄)₂HPO₄ | 36 |
| NH₄Cl | 31 |
| PTSA | 37 |
| (para-toluene sulfuric acid) | |

### EXAMPLE 2

This example demonstrates the effect of various additives on the curing reactions of sucrose.

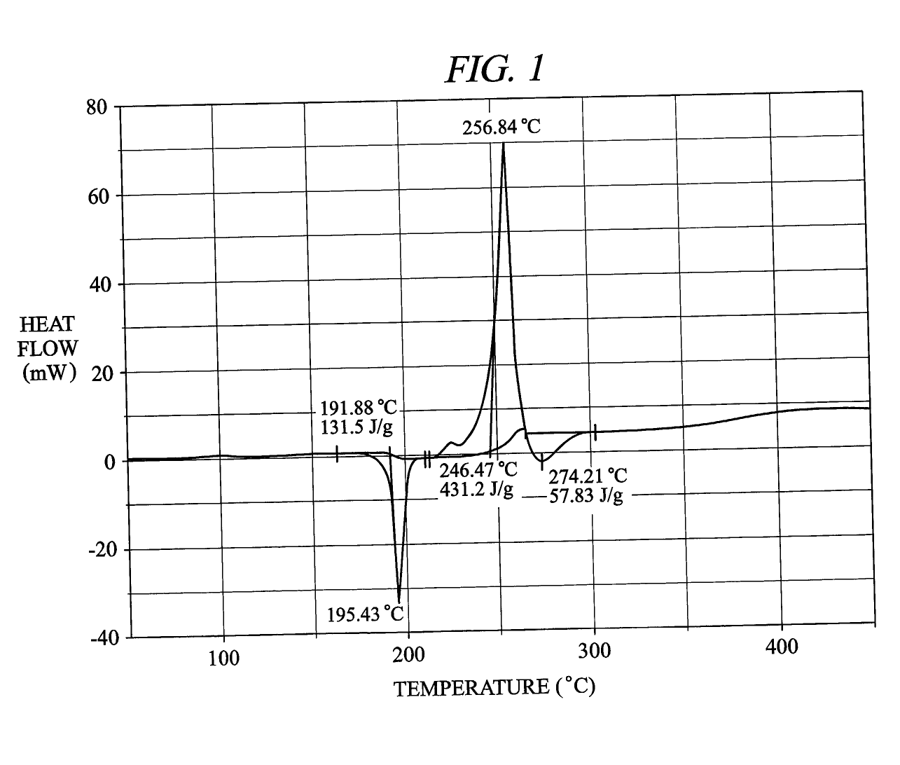

The curing process for sugars can be demonstrated using thermal analysis techniques: differential scanning calorimetry (DSC) and thermogravimetric analysis (TGA). FIG. 1 shows a DSC curve for sucrose without any additive. The DSC was performed using a pressure cell maintained at 800 psi argon pressure and a heating rate of 10° C./minute. The use of pressure reduces the effects of volatilization to more clearly define the reaction exotherm. The TGA was carried out at atmospheric pressure in an argon atmosphere at a heating rate of 10° C./minute.

The DSC curve shown in FIG. 1 for sucrose without additive exhibits an endothermic peak at about 195° C. for melting of the sucrose and an exothermic peak at about 256° C. resulting from the curing reaction.

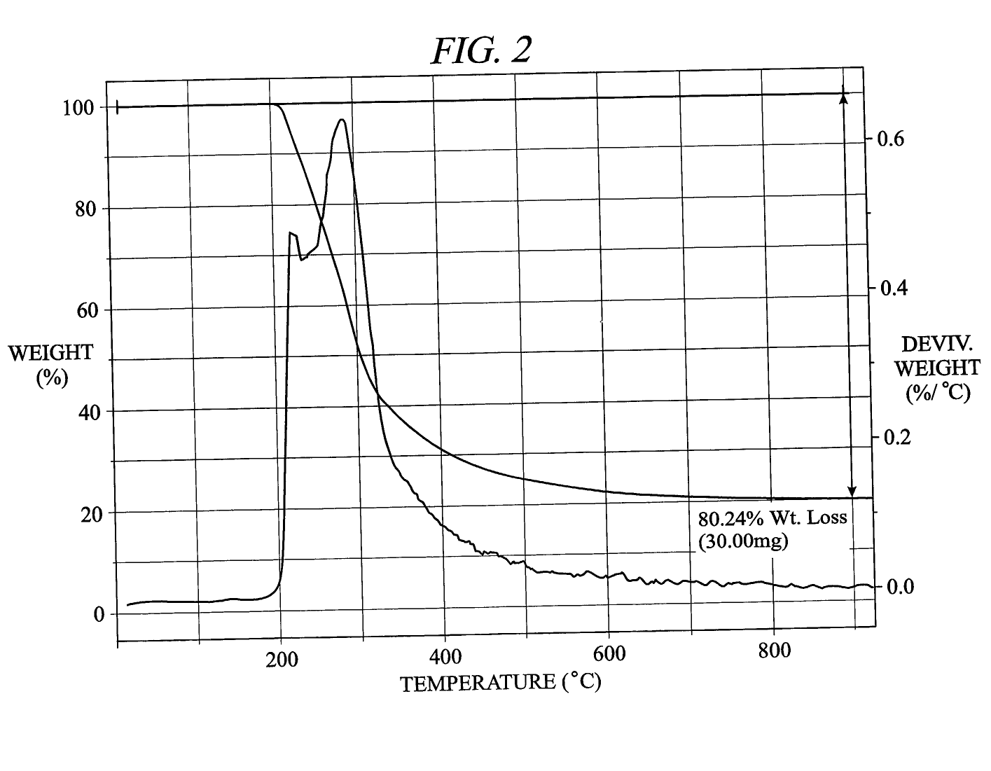

The TGA curve for sucrose, shown in FIG. 2, shows that weight loss commences about 200° C. and continues during the exothermic polymerization process. The final TGA carbon yield is 19.8%.

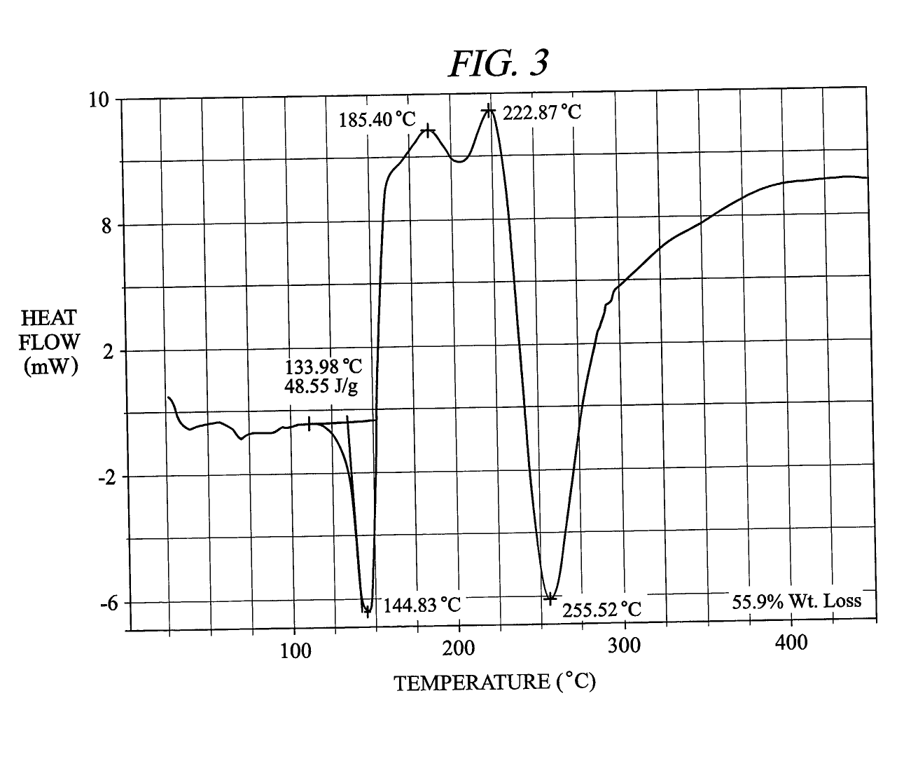

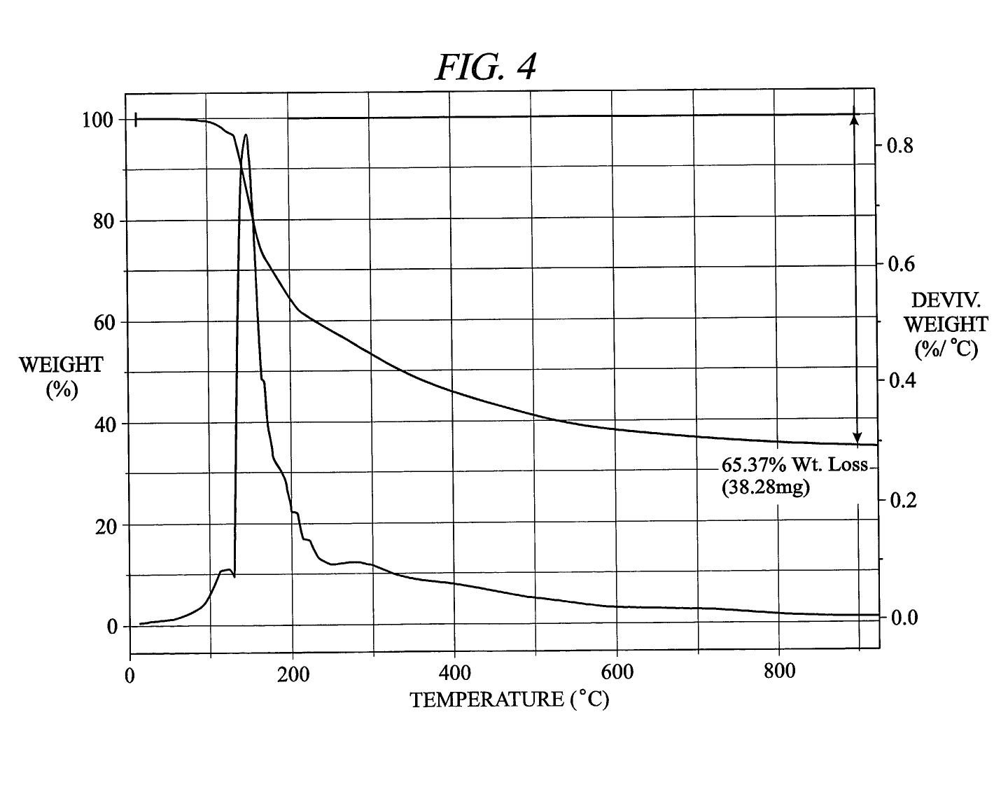

FIG. 3 shows a DSC curve for sucrose containing 3.7% of ammonium dihydrogen phosphate as an additive. There is an endothermic peak at 144° C. followed by two exothermic peaks at about 185° C. and 222° C. The TGA curve for the same blend in FIG. 4 shows a weight loss onset at above 100° C. with the major weight loss peak at about 140° C. This initial weight loss occurs before the major polymerization. The NH₄H₂PO₄ additive catalyzes a low temperature reaction of sucrose and lead to a more gradual evolution of volatile water. Chemical analysis of the reaction residues indicates the following staged reaction for sucrose with additive.

140° C. C₁₂H₂₂O₁₁→C₁₂H₁₄O₇+4H₂O

180-250° C. C₁₂H₁₄O₇→C₁₂H₈O₄+3H₂O

The low temperature reaction is seen to stabilize the carbon in the sucrose and allow its retention during carbonization. The carbon residue resulting from the run demonstrated in FIG. 4 is 34.6%.

Similar effects were demonstrated for other additives investigated including, ammonium monohydrogen phosphate, ammonium chloride and PTSA. Table II lists the exotherm peak temperatures from DSC and the onset of weight loss temperatures from TGA for sucrose containing 3.7% of these additives.

TABLE II

| | Exothermic Peak ° C. | Wt. Loss Onset |
|---|---|---|
| Additive | (DSC) | Temp ° C. (TGA) |
| None | 257 | 210 |
| NH₄H₂PO₄ | 185,220 | 140 |
| (NH₄)₂H₂PO₄ | 184,230 | 145 |
| NH₄Cl | 184,222 | 150 |
| PTSA | 144,192 | 120 |

For each additive the carbon yield was increased by over 50% from that of sucrose alone and the derived carbon was hard and dense compared to sucrose carbon.

### EXAMPLE 3

This example demonstrates the effects of additives on the curing of fructose and glucose.

A 50% solution of the monosaccharide fructose in water was prepared by combining 50 grams of fructose and 50 grams of water. To a portion of this solution, ammonium chloride was added at a level of 2 parts ammonium chloride to 50 parts fructose. The viscosity of this solution at room temperature was about 10 cps, indicating it was suitable for use as an impregnant. Next, water was removed from the solution by heating under vacuum at about 70° C. MCC measurements for the residues showed an expected 21% for the fructose alone and 37% for the fructose containing 4% of ammonium chloride.

A similar example was carried out using the monosaccharide glucose. Aqueous solutions containing 40% glucose both with and without the addition of 4 parts of ammonium chloride were prepared. Following removal of water, the glucose residue as expected had an MCC of 20%, while the glucose with additive had an MCC of 31%.

The ammonium chloride also altered the reaction temperature for polymerization and weight loss for both glucose and fructose as shown by the results in Table III.

TABLE III

| | Exothermic Peak ° C. | Wt. Loss Onset |
|---|---|---|
| Sugar System | (DSC) | Temp ° C. (TGA) |
| Fructose | 242 | 170 |
| Fructose + 4% NH₄Cl | 172,220 | 135 |
| Glucose | 290 | 200 |
| Glucose + 4% NH₄Cl | 184,222 | 150 |

### EXAMPLE 4

Blends of fructose containing 0, 2, 3, and 4% of ammonium chloride were prepared by mixing the solid components at room temperature. As in Example 3, the blends were characterized by MCC measurement, DSC and TGA. The results in Table IV show that even at the lower 2% level, the ammonium chloride additive increased the MCC and reduced the reaction temperature for loss of water and exothermic curing.

TABLE IV
Effect of Ammonium Chloride and Carbonization of Fructose

| Addition Level of | MCC % | TGA Wt. Loss | |
|---|---|---|---|
| NH₄Cl (%) | Onset ° C. | Exotherm ° C. | Major |
| 0 | 20 | 170 | 242 |
| 2 | 29 | 135 | 186 |
| 3 | 32 | 130 | 182 |
| 4 | 32 | 130 | 179 |

What is claimed is:

1. A carbon precursor composition comprising a sugar and containing as an additive one or more reactive additives materials in a amount sufficient to lower the melting point and exothermic polymerization temperatures of the said sugar.

2. The composition of claim 1 wherein the additive comprises an acid or acid salt.

3. The composition of claim 1 wherein the additive is selected from the group consisting of a phosphate; ammonium chloride; zinc chloride; aluminum chlorate; or para-toluene sulfonic acid.

4. The composition of claim 3 wherein the phosphate comprises ammonium dihydrogen phosphate and ammonium monohydrogen phosphate.

5. The composition of claim 3 wherein the additive is present in an amount of up to about 6% by weight, based on the weight of the sugar.

6. The composition of claim 1 wherein the said sugar and the said additive are dissolved in water to form an aqueous solution.

7. A process for increasing the mechanical strength and decreasing the porosity of a carbonaceous material comprising the steps of (1) coating the carbonaceous material with a molten mixture of a sugar and one or more reactive additives sufficient to lower the melting point and exothermic polymerization temperature of the sugar and (2) baking said carbonaceous material for a time and at a temperature sufficient to convert at least about 75% of the carbon content of the sugar into a pore filling carbonaceous residue.

8. The process of claim 7 wherein the additive comprises an acid or acid salt.

9. The process of claim 7 wherein the additive is selected from the group consisting of a phosphate; ammonium chloride; zinc chloride; aluminum chlorate; or para-toluene sulfonic acid.

10. The process of claim 9 wherein the phosphate comprises ammonium dihydrogen phosphate and ammonium monohydrogen phosphate.

11. The process of claim 9 wherein the additive is present in an amount of up to about 6% by weight, based on the weight of the sugar.

12. The process of claim 7 wherein the said sugar and the said additive are dissolved in water to form an aqueous solution.

13. A carbonaceous material with increased mechanical strength prepared according to the process of claim 7.

14. A carbonaceous product prepared by heating a carbonaceous source material together with a binder comprising a sugar and a reactive additive in a quantity sufficient to lower the melting point and exothermic polymerization temperature of the sugar, until the carbonaceous source material and binder are formed into an integral carbonaceous material, with at least about 75% of the initial intrinsic carbon content of the sugar being incorporated into the integral carbonaceous material.

15. The product of claim 14 wherein the additive comprises an acid or acid salt.

16. The product of claim 14 wherein the additive is selected from the group consisting of a phosphate; ammonium chloride; zinc chloride; aluminum chlorate; or para-toluene sulfonic acid.

17. The product of claim 16 wherein the phosphate comprises ammonium dihydrogen phosphate and ammonium monohydrogen phosphate.

18. The product of claim 16 wherein the additive is present in an amount of up to about 6% by weight, based on the weight of the sugar.

19. The product of claim 14 wherein the said sugar and the said additive are dissolved in water to form an aqueous solution.
